(19) United States
(12) Patent Application Publication
(10) Pub. No.: US 2026/0260755 A1
(43) Pub. Date: Sep. 3, 2026

(54) ASSISTANCE METHOD, ASSISTANCE DEVICE, AND RECORDING MEDIUM

(71) Applicant: Panasonic Intellectual Property Management Co., Ltd., Osaka (JP)

(72) Inventors: Maho SHIOTANI, Osaka (JP); Katsuhisa Yamaguchi, Osaka (JP); Miwa Takewa, Osaka (JP)

(21) Appl. No.: 19/650,646

(22) Filed: Apr. 17, 2026

Related U.S. Application Data
(63) Continuation of application No. PCT/JP2024/033833, filed on Sep. 24, 2024.

(30) Foreign Application Priority Data
Oct. 31, 2023 (JP) 2023-186655

(51) Int. Cl. G16H 50/20; G06F 16/34

(52) U.S. Cl. CPC G16H 50/20; G06F 16/345

(57) ABSTRACT

An assistance method is an assistance method for assisting a user to know the health condition of a subject, and includes: obtaining a care record of the subject in a first period, where the care record includes text data; determining whether the subject has an abnormal condition based on the text data, using a natural language processing model; and outputting presentation information to the information terminal of the user, where the presentation information is based on a result of the determining.

### CROSS REFERENCE TO RELATED APPLICATIONS

This is a continuation application of PCT International Application No. PCT/JP2024/033833 filed on September 24, 2024, designating the United States of America, which is based on and claims priority of Japanese Patent Application No. 2023-186655 filed on October 31, 2023. The entire disclosures of the above-identified applications, including the specifications, drawings and claims are incorporated herein by reference in their entirety.

### DESCRIPTION

### FIELD

The present disclosure relates to assistance methods, assistance devices, and recording media for assisting users to know the health conditions of subjects.

### BACKGROUND

The 2025 problem in Japan is an aging society problem in which all eight million people belonging to what is called "the baby boomer generation" will reach the ages of 75 or older, resulting in a quarter of the nation's population reaching the ages of 75 or older. This problem involves a problem of labor shortage caused by increasing demands for healthcare and caregiving services.

It is therefore demanded to reduce the load of users such as healthcare and caregiving workers who are in charge of subjects including those being nursed and cared for. For example, Patent Literature (PTL) 1 discloses a system for automatically generating care diaries (care records) from voice input.

### Citation List

### Patent Literature

PTL 1: Japanese Unexamined Patent Application Publication No. 2022-120752

### SUMMARY

### Technical Problem

The system disclosed in PTL 1, however, merely generates care records automatically, and the users are required to check care records to know the health conditions of subjects, resulting in limited effects of reducing the loads of the users.

In view of this, the present disclosure provides an assistance method, an assistance device, and a recording medium capable of assisting users to know the health conditions of subjects.

### Solution to Problem

An assistance method according to one aspect of the present disclosure is an assistance method for assisting a user to know the health condition of a subject, and includes: obtaining a care record of the subject in a first period, where the care record includes text data; determining whether the subject has an abnormal condition based on the text data, using a natural language processing model; and outputting presentation information to the information terminal of the user, where the presentation information is based on a result of the determining.

An assistance device according to one aspect of the present disclosure is an assistance device for assisting a user to know the health condition of a subject, and includes: an obtainer that obtains a care record of the subject in a first period, where the care record includes text data; a determiner that determines whether the subject has an abnormal condition based on the text data, using a natural language processing model; and an outputter that outputs presentation information to the information terminal of the user, where the presentation information is based on a result of the determination.

A recording medium according to one aspect of the present disclosure is a non-transitory computer-readable recording medium for use in a computer, the recording medium having recorded thereon a computer program for causing the computer to execute the aforementioned assistance method.

### Advantageous Effects

With the assistance method and others according to the present disclosure, it is possible to achieve, for instance, an assistance method capable of assisting the users to know the health conditions of subjects.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a diagram illustrating one example of the configuration of a physical condition detection system according to an embodiment.

FIG. 2 is a block diagram illustrating one example of the functional configuration of an information management server according to an embodiment.

FIG. 3 is a diagram illustrating examples of a care record according to an embodiment.

FIG. 4 is a diagram for explaining a process performed by a calibration processor according to an embodiment.

FIG. 5 is a diagram illustrating state classification results obtained by a state classifier according to an embodiment.

FIG. 6 is a flowchart illustrating a first operation performed by the information management server according to an embodiment.

FIG. 7 is a diagram illustrating the original text of a care record according to an embodiment.

FIG. 8 is a diagram illustrating attention values assigned on a per part basis according to an embodiment.

FIG. 9 is a diagram schematically illustrating the calculation results of the attention values of each text data according to an embodiment.

FIG. 10A is a flowchart illustrating a first example of detailed operation performed in step S13 shown in FIG. 6.

FIG. 10B is a flowchart illustrating a second example of the detailed operation performed in step S13 shown in FIG. 6.

FIG. 11 is a diagram illustrating the attention value of parts according to an embodiment.

FIG. 12 is a flowchart illustrating detailed operation performed in step S15 shown in FIG. 6.

FIG. 13 is a diagram showing one example of a five-level graded scores and conditions for the graded scores according to an embodiment.

FIG. 14 is a diagram illustrating one example of a daily summary including a score according to an embodiment.

FIG. 15 is a diagram illustrating one example of a weekly summary according to an embodiment.

FIG. 16 is a diagram illustrating one example of a weekly summary including activity data according to an embodiment.

FIG. 17 is a flowchart illustrating a second operation performed by the information management server according to an embodiment.

FIG. 18 is a diagram illustrating a first example of aggregate information according to an embodiment.

FIG. 19 is a diagram illustrating a second example of the aggregate information according to an embodiment.

FIG. 20 is a diagram illustrating a third example of the aggregate information according to an embodiment.

FIG. 21 is a block diagram illustrating one example of the functional configuration of a model generation device according to a variation of an embodiment.

FIG. 22 is a flowchart illustrating an operation of generating a machine learning model according to the variation of an embodiment.

### DESCRIPTION OF EMBODIMENTS

An assistance method according to a first aspect of the present disclosure is an assistance method for assisting a user to know the health condition of a subject, and includes: obtaining a care record of the subject in a first period, where the care record includes text data; determining whether the subject has an abnormal condition based on the text data, using a natural language processing model; and outputting presentation information to the information terminal of the user, where the presentation information is based on a result of the determining.

Since whether a subject has an abnormal condition can be determined automatically and output to the information terminal of a user, the user can know the health condition of the subject by just viewing information presented on the information terminal of the user. For example, the user can know the health condition of the subject without checking care records of the user. With the assistance method according to the present disclosure, it is possible to assist the user to know the health condition of the subject.

For example, an assistance method according to a second aspect of the present disclosure is the assistance method according to the first aspect, and the assistance method may further include determining, when the subject has an abnormal condition, the type of the abnormal condition based on the text data, using the natural language processing model. The presentation information may include information based on the type of the abnormal condition determined.

Since the type of an abnormal condition of the subject can be determined automatically, the user can know in more details the health condition of the subject just by viewing information presented on the information terminal of the user. With the assistance method according to the present disclosure, it is possible to assist the user to know the health condition of the subject.

For example, an assistance method according to a third aspect of the present disclosure is the assistance method according to the first or second aspect, and the assistance method may further include: extracting at least one of the following from the care record, using a learning model: (i) a normal section describing that the subject is in normal condition; or (ii) an abnormal section describing that the subject is in abnormal condition; and generating a first summary describing the health condition of the subject in the first period, based on the at least one of the normal section or the abnormal section extracted. The presentation information may include the first summary.

This enables the user to know in detail the health condition of the subject in a first period just by reading a first summary, i.e., without reading care records of the subject.

For example, an assistance method according to a fourth aspect of the present disclosure is the assistance method according to the third aspect, and in the generating of the first summary: the text data may be divided into parts; the attention value of each of the parts may be calculated, where the attention value indicates a degree to which the part contributes to a result of the determining of the type of the abnormal condition; and the first summary may be generated based on the attention values of the parts.

With this, it is possible to generate a first summary in accordance with the result of determining whether the subject has an abnormal condition.

For example, an assistance method according to a fifth aspect of the present disclosure is the assistance method according to the fourth aspect, and in the generating of the first summary, at least one part whose attention value is a first predetermined value may be extracted from among the parts, and the first summary may be generated based on the at least one part extracted.

With this, when the subject has an abnormal condition, it is possible to generate a first summary including a part related to the abnormal condition. With such a first summary being presented, the user can know the health condition of the subject more efficiently.

For example, an assistance method according to a sixth aspect of the present disclosure is the assistance method according to the fifth aspect, and the assistance method may further include: obtaining activity data of the subject in the first period; calculating a feature based on the activity data obtained; and obtaining, based on the feature, an abnormal score indicating a degree of the abnormal condition per first period. The presentation information may include information that associates the first summary with a score that is based on the abnormal score.

This allows the meaning of a score to be interpreted based on a first summary. If only the score is presented, for example, there is a risk that the user may erroneously determine a high score as incorrect when the subject appears to be behaving normally. In contrast, in the present disclosure, since the first summary is presented together with the score, the user can know the reason for that score from the first summary, which can help prevent such erroneous determination.

For example, an assistance method according to a seventh aspect of the present disclosure is the assistance method according to the second aspect, and a second period may include two or more first periods each of which is the first period. The assistance method may further include: using a learning model, extracting at least one of the following from the care record: (i) a normal section describing that the subject is in normal condition; or (ii) an abnormal section describing that the subject is in abnormal condition; generating a first summary describing the health condition of the subject in the first period, based on the at least one of the normal section or the abnormal section extracted; and generating a second summary based on (i) two or more first summaries in each of the two or more first periods included in the second period, where each of the two or more first summaries is the first summary, and (ii) an attention value of each of the two or more first summaries, where the attention value indicates a degree to which the first summary contributes to the result of the determining of whether the subject has an abnormal condition. The presentation information may include the second summary.

This enables the user to know the health condition of the subject in a second period just by reading a second summary, i.e., without reading care records including a huge amount of records for the second period.

For example, an assistance method according to an eighth aspect of the present disclosure is the assistance method according to the seventh aspect, and in the generating of the second summary, at least one sentence whose attention value is a second predetermined value may be extracted from the two or more first summaries, and the second summary may be generated based on the at least one sentence extracted.

With this, it is possible to generate a second summary in accordance with the result of determining whether the subject has an abnormal condition.

For example, an assistance method according to a ninth aspect of the present disclosure is the assistance method according to the seventh or eighth aspect, and may further include: obtaining activity data of the subject obtained by sensing the subject in the second period; and outputting time-series data of the activity data to the information terminal of the user, where the time-series data is associated with the second summary.

This enables the user to refer to activity data to know the health condition of the subject. Therefore, by checking the activity data, the user can easily know the health condition of the subject.

For example, an assistance method according to a tenth aspect of the present disclosure is the assistance method according to any one of the first to ninth aspects, and the assistance method may further include: collecting a record of a specific abnormal condition among abnormal conditions associated with the subject; and aggregating occurrence dates and times of two or more specific abnormal conditions that have been collected. The presentation information may include information based on a result of the aggregating.

This can assist the user to know a tendency related to the occurrence date and time of a specific abnormal condition.

For example, an assistance method according to an eleventh aspect of the present disclosure is the assistance method according to the tenth aspect, and the information based on the result of the aggregating may include information indicating an influence of a season or time on occurrence of the specific abnormal condition.

This can assist the user to know an influence of a season or time on the occurrence of a specific abnormal condition.

For example, an assistance method according to a twelfth aspect of the present disclosure is the assistance method according to any one of the first to eleventh aspects, and the assistance method may further include extracting a description related to at least one of the following from the care record: a predetermined item; a predetermined activity of the subject; or a report on a consultation excluding a regular check-up. The text data may include the description extracted.

Since descriptions unnecessary for determining whether the subject has an abnormal condition are excluded, the accuracy of a determination result obtained by using a natural language processing model can be enhanced.

An assistance device according to a thirteenth aspect of the present disclosure is an assistance device for assisting a user to know the health condition of a subject, and includes: an obtainer that obtains a care record of the subject in a first period, where the care record includes text data; a determiner that determines whether the subject has an abnormal condition based on the text data, using a natural language processing model; and an outputter that outputs presentation information to the information terminal of the user, where the presentation information is based on a result of the determination. A recording medium according to a fourteenth aspect of the present disclosure is a non-transitory computer-readable recording medium for use in a computer, the recording medium having recorded thereon a computer program for causing the computer to execute the assistance method according to any one of the first to twelfth aspects of the present disclosure.

With this, the same advantageous effects as produced by the aforementioned assistance method are produced.

Note that these general or specific aspects may be implemented using a system, a method, an integrated circuit, a computer program, or a non-transitory computer-readable recording medium such as a CD-ROM, or any combination of systems, methods, integrated circuits, computer programs, and recording media. The program(s) may be stored in a recording medium in advance or may be supplied to a recording medium via a wide area communication network including, for instance, the Internet.

Hereinafter, exemplary embodiments of the present disclosure will be described in detail with reference to the drawings.

Note that each of the exemplary embodiments described below shows a general or specific example. The numerical values, constituent elements, the arrangement and connection of the constituent elements, steps, order of the steps etc. shown in the following exemplary embodiments are mere examples, and therefore do not intend to limit the present disclosure. Among the constituent elements in the following exemplary embodiments, those not recited in the independent claims will be described as optional constituent elements.

The drawings are represented schematically and are not necessarily precise illustrations. Thus, the scales, for example, are not necessarily consistent from drawing to drawing. In the drawings, constituent elements that are substantially the same are given the same reference signs, and redundant descriptions will be omitted or simplified.

In the present specification, numerical values and numerical value ranges do not express the strict meanings only, but also include substantially equivalent ranges, e.g., differences of several percent (or approximately 10%).

In the present specification, ordinal numerals such as "first" and "second" do not mean the number or order of constituent elements unless otherwise stated in particular. The ordinal numerals are used to avoid confusion of constituent elements of the same type and distinguish between the constituent elements.

### Embodiment

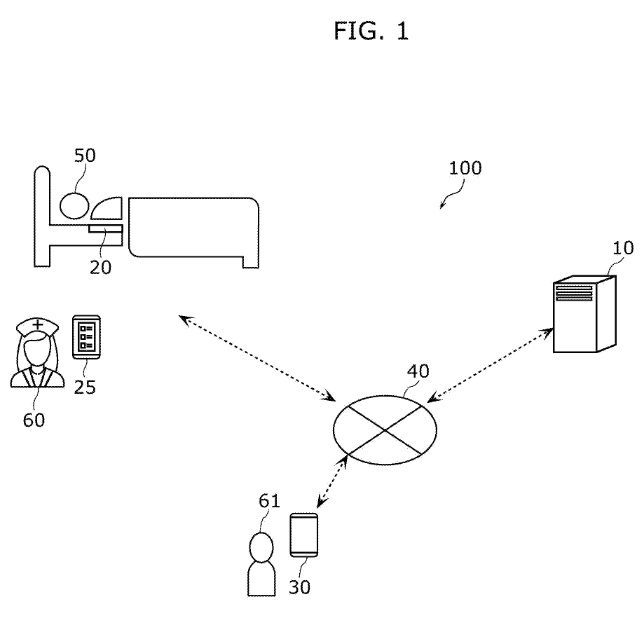

Hereinafter, an assistance method and others according to the present embodiment will be described with reference to FIG. 1 to FIG. 20.

### 1. Configuration of physical condition detection system

First, the configuration of a physical condition detection system including an information management server that executes an assistance method according to the present embodiment will be described with reference to FIG. 1 to FIG. 5. FIG. 1 is a diagram illustrating one example of the configuration of physical condition detection system 100 according to the present embodiment.

Physical condition detection system 100 according to the present embodiment is a system configured for information management server 10 to assist the user to know the health condition of subject 50 who is nursed or cared for.

As illustrated in FIG. 1, physical condition detection system 100 includes information management server 10, sensor 20, care record collector 25, and display terminal 30. These components are communicably connected to each other over communication network 40. Communication network 40 may be a wired network, a wireless network, or the both. FIG. 1 illustrates subject 50 who is nursed or cared for, user 60 who is an on-site staff member such as a healthcare worker providing nursing or care for subject 50, and user 61 who is an on-site staff member such as a supervisor of subject 50 and can check display terminal 30.

Note that FIG. 1 illustrates a case where physical condition detection system 100 includes one sensor 20, but the physical condition detection system according to the present disclosure is not limited to this example, and may include sensors 20 as many as the number of subjects 50 who are nursed or cared for.

Sensor 20 obtains activity data of subject 50 in a predetermined period through sensing. The activity data includes data related to the activity of subject 50. The activity data includes at least one of the following: heat rate, respiratory rate, in-bed or out-of-bed rate, body temperature, number of times turning over in bed, and food intake, and may include, for example, at least the heart rate. The activity data may include at least two of the following: heat rate, respiratory rate, in-bed or out-of-bed rate, body temperature, number of times turning over in bed, and food intake. For example, sensor 20 may obtain, on a per second basis, data such as the heart rate, respiratory rate, body movements of subject 50 while they are in bed (hereinafter also referred to as “sensor data”). Sensor 20 may sense subject 50 is in bed or out of bed based on whether heart rate, respiratory rate, and body movements of subject 50 can be sensed.

The interval for obtaining sensor data such as heart rate, respiratory rate, and body movements does not have to be every second, and may be, for example, every two seconds as long as it is a unit interval that allows changes in the sensor data of 50 subject to be detected. Sensor 20 may further sense daily routines such as sleep patterns based on whether heart rate, respiratory rate, and body movements can be sensed.

Sensor 20 may include, for example, a sensor that performs sensing related to excretion, and may sense the shape and color of the excreta, the time required for excretion, and so on.

Sensor 20 may be, for example, an imaging device such as a camera and is provided to be capable of capturing images of subject 50 in the bed or subject 50 who is eating. The camera may be a thermal camera that detects the body temperature of subject 50 or a normal camera (e.g., a charge coupled device (CCD) camera). Food intake refers to an amount of food taken by subject 50 at breakfast, lunch, or dinner, and may be obtained by performing image analysis on an image. Note that the body temperature and food intake may be obtained through input into care record collector 25 by user 60.

Hereinafter, a case where the activity data includes respiratory rate and heart rate will be mainly described.

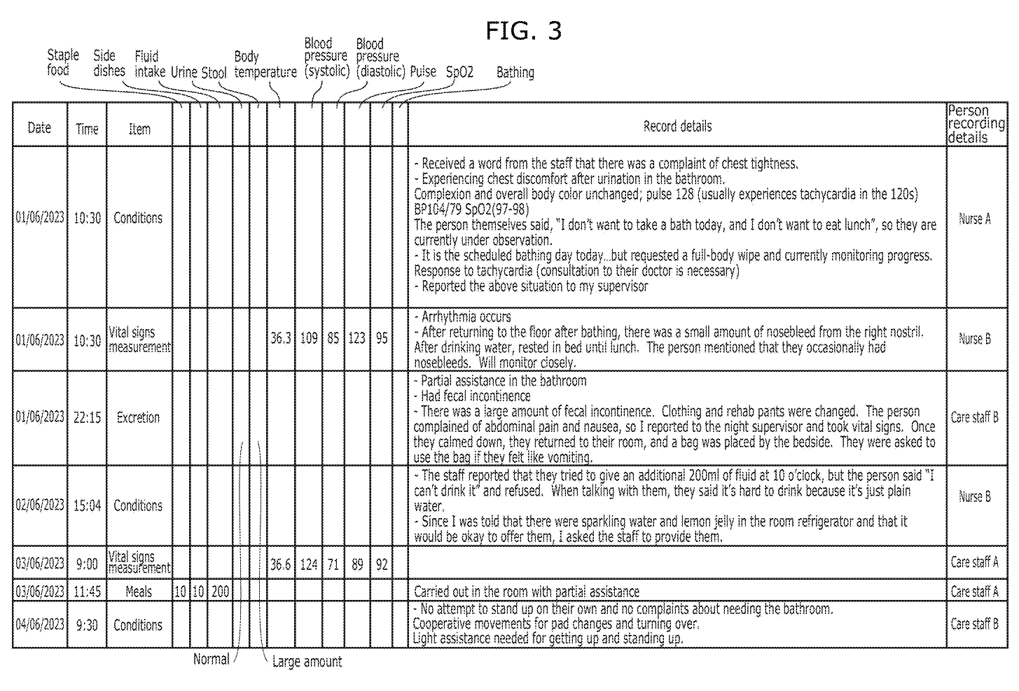

Care record collector 25 collects care records from user 60. The care records are each a record of details of nursing or care of subject 50 by user 60, changes in the condition of subject 50, and their living situation (for example, see FIG. 3 described later). Each care record includes free-form text data describing the condition and behavior of subject 50 when user 60 performs the nursing or care of subject 50.

Care record collector 25 includes an input unit that receives a care record from user 60 and a display that displays a screen on which the care record is input. The input unit is, for example, a touch panel, a keyboard, a sound collecting device (e.g., a microphone), but is not limited to these examples. The input unit is, for example, a display device such as a display, but is not limited to this example. Care record collector 25 may be a mobile terminal device such as a smartphone or a tablet, or a stationary terminal device such as a personal computer (PC).

Information management server 10 is implemented using, for example, a computer including a processor (a microprocessor), memory, a communication interface, and the like. Information management server 10 may be configured to operate with a part of the configuration of information management server 10 included in a cloud server. Information management server 10 is a device for assisting user 61 to know the health condition of subject 50 who is nursed or cared for. In other words, information management server 10 assists the monitoring of subject 50 performed by, for instance, user 61. Information management server 10 may also be a device that assists, for instance, user 61 in not overlooking small changes in the condition of subject 50 that could lead to an abnormal condition of subject 50 (i.e., early signs of an abnormal condition).

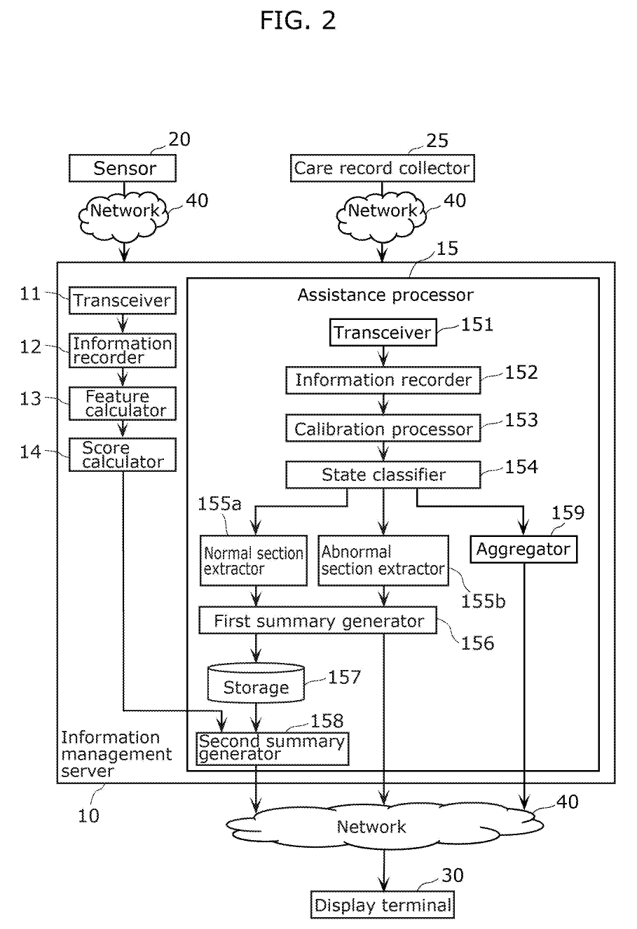

FIG. 2 is a block diagram illustrating one example of the functional configuration of information management server 10 according to the present embodiment.

As illustrated in FIG. 2, information management server 10 includes transceiver 11, information recorder 12, feature calculator 13, score calculator 14, and assistance processor 15. An assistance device is configured by including, for example, at least assistance processor 15. The assistance device may be implemented as a stand-alone device.

Transceiver 11 includes, for example, a communication interface and transmits and receives various types of information to and from sensor 20 or display terminal 30 over communication network 40. For example, transceiver 11 obtains activity data including the respiratory rate and heart rate of subject 50 in a predetermined period. Here, the activity data may include, for example, at least respiratory rate and heart rate among the body temperature, food intake, respiratory rate, heart rate, and out-of-bed rate of subject 50 in the predetermined period.

In the present embodiment, transceiver 11 obtains, from sensor 20 over communication network 40, sensor data such as the heart rate, respiratory rate, body movements per second of subject 50 while they are in bed, for example, at predetermined intervals of one minute. In this way, transceiver 11 obtains activity data that includes sensor data and can be obtained daily on-site through communication network 40. Transceiver 11 is one example of the obtainer.

Information recorder 12 records information that transceiver 11 transmits and receives. Information recorder 12 is a recording medium capable of recording information and includes non-volatile memory such as a hard disk drive or solid-state drive. Information recorder 12 may record a plurality of features calculated by feature calculator 13.

Feature calculator 13 includes a computer including, for example, memory and a processor (a microprocessor), and realizes the function of calculating a plurality of features by the processor executing a control program stored in the memory. Feature calculator 13 calculates a plurality of features based on activity data including the respiratory rate and heart rate of subject 50 that are obtained by transceiver 11. For example, feature calculator 13 obtains sensor data for a time period including target date and time of physical condition detection from activity data obtained by transceiver 11 or recorded on information recorder 12, and calculates an hourly feature, such as a respiratory rate, for each sensor data.

Feature calculator 13 may calculate, for example, at least the mean value and maximum value of the respiratory rates of subject 50, and the mean value and maximum value of the heart rates of subject 50, as a plurality of hourly features. Feature calculator 13 calculates, from at least the respiratory rates and the heart rates, the mean values and the maximum values of the respiratory rates and the heart rates, as the plurality of features, from among mean values, maximum values, standard deviations, skewnesses, kurtoses, and impulse factors of the respiratory rates, difference data on the respiratory rates, the heart rates, and difference data on the heart rates. Feature calculator 13 thus performs statistical processing and the like on the activity data and calculates the plurality of features.

More specifically, feature calculator 13 calculates, for example, respiratory-rate-related features and heart-rate-related features of subject 50 on an hourly basis. For example, feature calculator 13 obtains sensor data indicating the respiratory rates of subject 50 within a time period including target date and time of physical condition detection from activity data recorded on information recorder 12 or sensor data obtained from sensor 20, and calculates hourly statistical features for the time period.

More specifically, feature calculator 13 obtains, for example, respiratory rate data indicating respiratory rates that are not zero during a given hour from the activity data, and calculates statistical features such as the mean value, maximum value, minimum value, standard deviation, skewness, kurtosis, and impulse factor for that hour from the obtained respiratory rate data. Here, the impulse factor can be calculated from the difference between the maximum value and the mean value (maximum value − mean value) of the respiratory rate data for that hour. In addition, feature calculator 13 calculates statistical features such as the mean value, maximum value, minimum value, standard deviation, skewness, kurtosis, and impulse factor for that hour from the difference data of the obtained respiratory rate data. The difference data of the obtained respiratory rate data represents, for example, the difference between the respiratory rate at time t and the respiratory rate at time t+1 that is one second later, i.e., data indicating the difference in respiratory rate per second. Feature calculator 13 may calculate, as statistical features, at least the mean value and maximum value of the respiratory rates for that hour, from the obtained respiratory rate data.

For example, feature calculator 13 obtains heart rate data indicating the heart rates of subject 50 during the time period including the target date and time of physical condition detection from activity data recorded on information recorder 12 or sensor data obtained from sensor 20, and calculates hourly statistical features for that time period.

More specifically, feature calculator 13 obtains, for example, heart rate data indicating heart rates that are not zero during a given hour from the activity data, and calculates statistical features such as the mean value, maximum value, minimum value, standard deviation, skewness, kurtosis, and impulse factor for that hour from the obtained heart rate data. Additionally, feature calculator 13 calculates statistical features such as the mean value, maximum value, minimum value, standard deviation, skewness, kurtosis, and impulse factor for the that hour from the difference data of the obtained heart rate data. The difference data of the obtained heart rate data, like the difference data of the respiratory rate data, represents, for example, the difference between the heart rate at time t and the heart rate at time t+1 that is one second later, i.e., data indicating the difference in heart rate per second. Feature calculator 13 may calculate, as statistical features, at least the mean value and maximum value of the heart rates for that hour, from the obtained heart rate data.

Feature calculator 13 may calculate the food intake or out-of-bed rate of subject 50 as one of the plurality of features.

Score calculator 14 obtains an abnormal score indicating the degree of an abnormal condition per predetermined period based on the features calculated by feature calculator 13. For example, score calculator 14 obtains an abnormal score by inputting the plurality of features calculated by feature calculator 13 into a trained model (a supervised model) generated through supervised training.

Subsequently, score calculator 14 calculates a graded score that indicates, in stages, the degree of the abnormal condition of subject 50, based on the obtained abnormal score. The graded score is one example of a score based on an abnormal score.

In the present embodiment, score calculator 14 calculates a mean abnormal score per day from hourly abnormal scores on the target day of physical condition detection. Score calculator 14 likewise calculates the mean abnormal score per day the day before and two days before the target day of physical condition detection from hourly abnormal scores for the day before and two days before the target day. Score calculator 14 sums up the daily abnormal score for the target day, the daily abnormal score for the day before the target day, and the daily abnormal score for two days before the target day, and thus calculates a three-day total score. Note that the three-day total score is just one example of a calculation method for accurately deriving graded scores, and the calculation method is not limited to this example. The graded scores may be calculated over a range of one-day to five-day total scores.

Score calculator 14 calculates a threshold for a graded score (referred to as a stepped threshold in some cases) from a group of three-day total scores for approximately the past 90 days of the target date. More specifically, score calculator 14 calculates the stepped threshold by calculating the mean and standard deviation of the group of three-day total scores for approximately the past 90 days.

When score calculator 14 calculates a graded score that is, for example, at five levels, and the calculated graded score is 4 or 5, the calculated graded score may be output to display terminal 30 over communication network 40.

In this way, score calculator 14 quantifies changes in the physical condition of subject 50, and by presenting a score to user 61, it is possible to prevent subject 50 from becoming seriously ill due to overlooked changes in their condition.

Assistance processor 15 includes a computer including, for example, memory and a processor (a microprocessor), and realizes the function of generating data for assisting the monitoring of subject 50 performed by, for instance, user 61 by the processor executing a control program stored in the memory.

Assistance processor 15 includes transceiver 151, information recorder 152, calibration processor 153, state classifier 154, normal section extractor 155a, abnormal section extractor 155b, first summary generator 156, storage 157, second summary generator 158, and aggregator 159.

Transceiver 151 includes, for example, a communication interface, and transmits and receives various types of information between care record collector 25 and display terminal 30 over communication network 40. For example, transceiver 151 obtains care records of subject 50 in a predetermined period.

In the present embodiment, transceiver 151 obtains, on an hourly or daily basis, a care record including text data that indicates, for instance, the details of nursing or care of subject 50 provided by user 60 who is an on-site staff as shown in FIG. 1, and the condition of subject 50, from care record collector 25 over communication network 40. In this way, transceiver 151 obtains a care record including text data and obtained on-site on a daily basis through communication network 40. Transceiver 151 functions as an obtainer.

Note that the interval at which transceiver 151 obtains a care record is not limited to one hour or one day.

Transceiver 151 outputs information (e.g., presentation information) generated by first summary generator 156, second summary generator 158, and aggregator 159 to display terminal 30 of user 61. Transceiver 151 functions as an outputter.

Here, a care record obtained by transceiver 151 will be described with reference to FIG. 3. FIG. 3 is a diagram illustrating examples of a care record according to the present embodiment.

As illustrated in FIG. 3, a care record includes date, time, items, staple food, side dishes, fluid intake, urine, stool, blood pressure (systolic/diastolic), pulse, SpO2, bathing, record details, and a person recording the details. A care record contains numerical values such as body temperature as well as textual data such as record details. The record details are free-form text data.

Assistance processor 15 according to the present embodiment uses only text data out of numerical values and text data included in a care record in the generation of presentation information. Stated differently, assistance processor 15 does not use numerical values in the generation of presentation information.

Referring again to FIG. 2, information recorder 152 records information transmitted and received by transceiver 151. Information recorder 152 is a recording medium capable of recording information, and includes, for example, non-volatile memory such as a hard disk drive or solid-state drive. Information recorder 152 may record various types of models that processing units such as calibration processor 153, state classifier 154, normal section extractor 155a, and abnormal section extractor 155b use.

Calibration processor 153 is a processing unit for performing proofreading of text data included in a care record. Since a care record may contain typographical errors, calibration processor 153 corrects such errors. This can prevent a decline in the accuracy of a process, such as status classification, caused by typographical errors.

Calibration processor 153 performs proofreading of text data using language models. For example, a language model such as n-gram may be used.

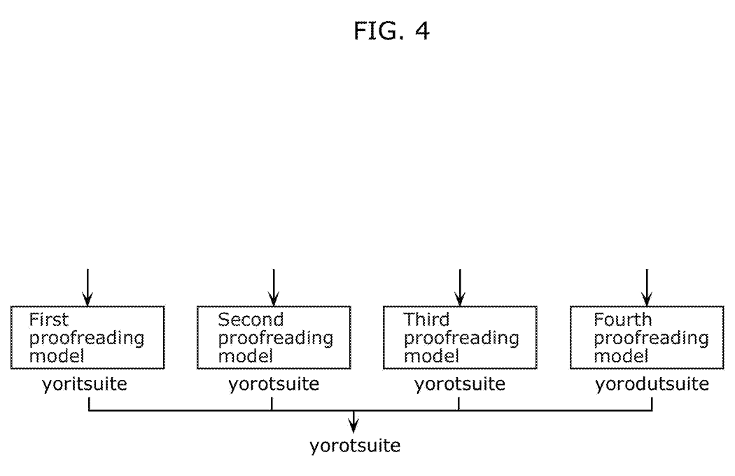

FIG. 4 is a diagram for explaining a process performed by calibration processor 153 according to the present embodiment.

As shown in FIG. 4, calibration processor 153 performs proofreading using a plurality of language models. In the present embodiment, calibration processor 153 uses four mutually different language models of a first proofreading model to a fourth proofreading model. Calibration processor 153 inputs the same text data to each of the four language models and obtains the proofread text data. FIG. 4 shows an example in which the data “yoritsuite” is output from the first proofreading model, the data “yorotsuite” is output from the second proofreading model and the third proofreading model, and the data “yorodutsuite” is output from the fourth proofreading model.

Calibration processor 153 determines the proofread text data by taking a majority vote of the outputs from the four language models. In the example in FIG. 4, since the data “yorotsuite” is output most frequently, calibration processor 153 determines that the correct expression in the corresponding part of the original text data is “yorotsuite” and corrects the part accordingly.

The number of language models used by calibration processor 153 may be one or more. Calibration processor 153 may, for example, input the same text data to one language model plural times, and determine the proofread text data by taking a majority vote of outputs obtained a predetermined number of times that is three or more. The same text data may be input plural times to at least one of the first proofreading model through the fourth proofreading model shown in FIG. 4.

Referring again to FIG. 2, state classifier 154 uses a natural language processing model to determine, based on the text data included in the care record of subject 50, whether subject 50 has an abnormal condition at least per first period. A natural language processing model is a model that outputs the presence or absence of an abnormal condition of subject 50 in the first period when the text data of subject 50 in the first period is input. The first period is, for example, one day, but is not limited to this example and may be, for instance, one hour. The first period may be, for example, set by user 61. State classifier 154 is one example of a determiner, and a natural language processing model is one example of a state classification model.

In the present embodiment, state classifier 154 also outputs a result of classifying an abnormal condition. The result includes an abnormal label that is an output of the natural language processing model. Abnormal labels indicating the classification (types) of abnormal conditions include restless, pain, dizziness, trauma, fever, gastrointestinal symptoms, movement deterioration, other incidents, respiratory issues, poor physical condition, edema, swallowing difficulties, drowsiness, intravenous infusion, cold symptoms, other infections, urinary issues, and so on. Hereinafter, abnormal labels may also be referred to as labels or inference labels.

Upon receiving the input of text data, state classifier 154 as such outputs a classification result indicating that the text data indicates a normal condition, or an abnormal condition when the text data indicates an abnormal condition. The natural language processing model is trained in advance through machine learning, using text data as input data and using, as correct-answer data, a classification result indicating that the text data indicates a normal condition, or a classification result indicating that the text data indicates an abnormal condition when the text data indicates an abnormal condition.

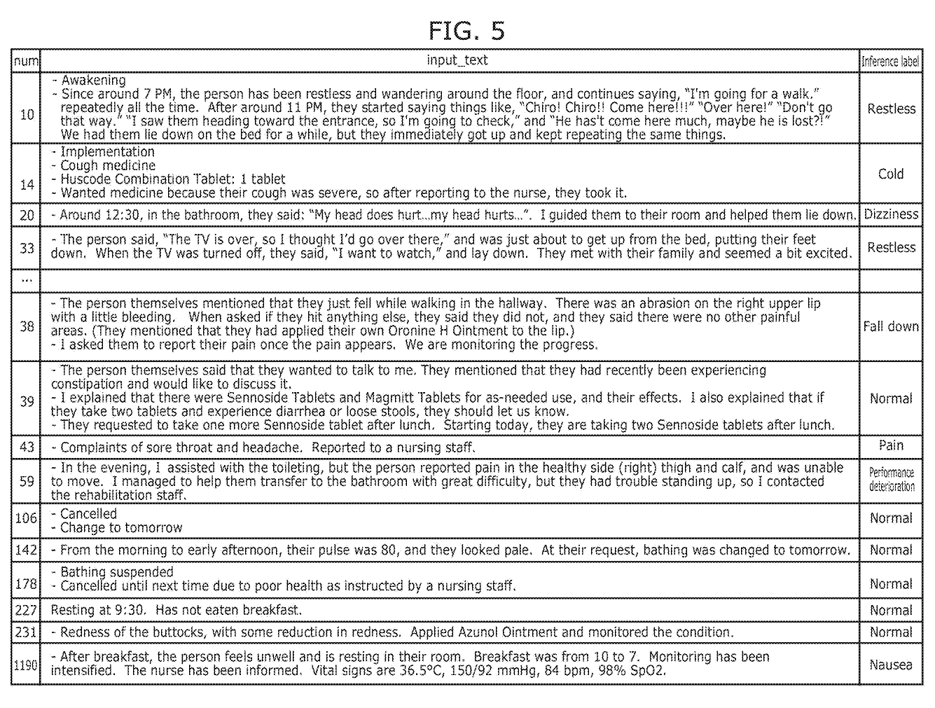

FIG. 5 is a diagram illustrating state classification results obtained by state classifier 154 according to the present embodiment.

As illustrated in FIG. 5, a state classification result includes No. (“num” in FIG. 5), text data input into the natural language processing model (“input_text” in FIG. 5), and the classification result of the natural language processing model (“inference label” in FIG. 5).

In the example in FIG. 5, for example, when the text data of number “10” is input into the natural language processing model, the classification result “restless” is output, and when the text data of number “14” is input into the natural language processing model, the classification result “cold” is output. When the text data of number “39” is input into the natural language processing model, the classification result “normal” is output.

When the natural language processing model determines only whether a subject has an abnormal condition, all parts except “normal” in the inference labels in FIG. 5 are considered “abnormal”.

State classifier 154 uses a natural language processing model generated in advance through supervised training. As a natural language processing model, existing models that correspond to the language used in the implementing facility (e.g., nursing care facilities or medical facilities) are used. For example, UTH-BERT, BERT pretrained in Japanese (Japanese version BERT), or RoBERTa (Robustly Optimized BERT Pretraining Approach) may be used as a natural language processing model, but the natural language processing model is not limited to this example.

In the present embodiment, the Japanese version of BERT is used as a natural language processing model. The Japanese BERT is created by training it on tasks such as predicting hidden words resulting from masking, using data like the “Wikipedia Cirrussearch” as of August 31, 2020 (approximately 17 million entries). For importing methods, “BertForSequenceClassification.from_pretrained(cl-tohoku/bert-base-japanese)” and “AutoTokenizer.from_pretrained(cl-tohoku/bert-base-japanese)” are used, but the methods are not limited to these. The model structure of the Japanese BERT adopts the structure used in general BERT models, and is, for example, “12 layers, hidden layer dimension of 768, 12 attention heads”.

In this way, state classifier 154 is configured to be capable of automatically determining whether a classification result indicates that subject 50 is in normal condition, or abnormal condition when subject 50 is in abnormal condition, based on text data through natural language processing.

Referring again to FIG. 2, sentences determined as describing normal conditions by state classifier 154 are input to normal section extractor 155a, and normal section extractor 155a summarizes the input sentences. When the sentences are input, normal section extractor 155a uses a machine learning model (a normal summary output model) that outputs a normal summary that summarizes the sentences. The machine learning model is trained in advance. The machine learning model may be an existing model.

A normal summary can be a sentence in which no abnormal condition of subject 50 is perceived. A normal summary includes, for example, information indicating that subject 50 appears to be enjoying themselves.

For example, mt5_summarize_japanese is used as a normal summary output model, but the normal summary output model is not limited to this example and may be, for example, BART, BERT, or LexRank.

Sentences determined as describing abnormal conditions by state classifier 154 are input to abnormal section extractor 155b, and abnormal section extractor 155b summarizes the input sentences. When the sentences are input, abnormal section extractor 155b uses a machine learning model (an abnormal section summary model) that extracts a sentence related to an abnormal condition from the sentences, to output an abnormal summary. The machine learning model is trained in advance. The machine learning model may be an existing model.

An abnormal summary can be a sentence that includes details regarding an abnormal condition of subject 50. For example, an abnormal summary includes information regarding abnormal labels that have been assigned. An abnormal section summary model is configured to be capable of specifying a sentence describing a significant abnormal condition among sentences and outputting an abnormal summary including the specified sentence. A sentence describing a significant abnormal condition is a sentence with a high attention value.

In this way, assistance processor 15 is configured to be capable of generating at least one of: a normal summary including a sentence describing that subject 50 is in normal condition; or an abnormal summary including a sentence describing that subject 50 is in abnormal condition.

First summary generator 156 generates a summary indicating the health condition of subject 50 in a first period, based on at least one of output from normal section extractor 155a or output from abnormal section extractor 155b. The summary may include details that are likely to be associated with one’s health or changes in the physical condition of subject 50. The summary indicates the overall physical condition of subject 50. First summary generator 156 generates a summary for each first period that is a relatively short period. In the present embodiment, first summary generator 156 generates a summary (a daily summary) each day. A summary generated by first summary generator 156 (e.g., a daily summary) is one example of the first summary.

First summary generator 156 generates a summary including a normal summary that is an output from normal section extractor 155a and an abnormal summary that is an output from abnormal section extractor 155b.

When two or more sentences are input, first summary generator 156 may use a machine learning model that generates a sentence joining the two or more sentences. The machine learning model is trained in advance. The machine learning model may be a language model. The machine learning model is, for example, mt5_summarize_japanese, but is not limited to this example and may be, for example, BART, BERT, or LexRank.

Storage 157 is a storage device that stores a summary generated by first summary generator 156. Storage 157 is a recording medium capable of recording information, and includes, for instance, rewritable non-volatile memory such as a hard disk drive or solid-state drive.

Second summary generator 158 generates a summary for each second period that is longer than the first period, based on two or more summaries generated by first summary generator 156. The second period includes, for example, a plurality of first periods. For example, second summary generator 158 generates a second summary indicating the health condition of subject 50 in a second period, based on (i) two or more first summaries of two or more first periods included in the second period and (ii) the attention value of each of the two or more first summaries (information indicating the level of attention). Attention values will be described later.

In the present embodiment, second summary generator 158 generates a summary (a weekly summary) each week. Second summary generator 158 generates a weekly summary by extracting, from daily summaries for one week, a sentence or summary having an attention value greater than or equal to a predetermined value.

When two or more sentences are input, second summary generator 158 uses a machine learning model that generates a sentence joining the two or more sentences. The machine learning model is trained in advance. The machine learning model may be a language model. The machine learning model is, for example, mt5_summarize_japanese, but is not limited to this example and may be, for example, BART, BERT, or LexRank.

Second summary generator 158 may include, into a weekly summary, activity data of subject 50 obtained from sensor 20.

Aggregator 159 executes aggregation of abnormal labels based on the history of abnormal labels. Aggregator 159 collects the record of the same abnormal label as a target abnormal label (a target label) from the history of abnormal labels. The record includes at least information indicating the date and time when an abnormal condition indicated by the abnormal label was detected. For example, date and time based on a care record may be associated as the date and time when an abnormal label was detected. The target label is input by, for instance, user 61.

Aggregator 159 aggregates the date and time of occurrence of an abnormal condition indicated by the target label, based on the aggregated records. Aggregator 159 aggregates the number of occurrences for each predetermined period such as each time period (e.g., each morning, day, or night), each month, or each season. The aggregation is performed for each subject 50. This makes it possible to check the tendency of the occurrence of the abnormal condition indicated by the target label in subject 50. Presenting such information to user 61 can lead to the discovery of information regarding the physical condition of subject 50 that had previously gone unnoticed and was buried in care records.

Display terminal 30 is implemented by a computer including, for instance, a processor (a microprocessor), memory, a communication interface, and a user interface. Display terminal 30 is the terminal of user 61 such as the observer of subject 50, and is a mobile terminal device such as a tablet or a smartphone, but may be a desktop computer (a stationary terminal device) connected to a display. Display terminal 30 is one example of the information terminal.

Display terminal 30 is connected to communication network 40, and upon obtaining presentation information from information management server 10, causes the user interface to display the presentation information. The user interface includes a display device such as a liquid crystal display, but is not limited to this example.

### 2. Operation of information management server

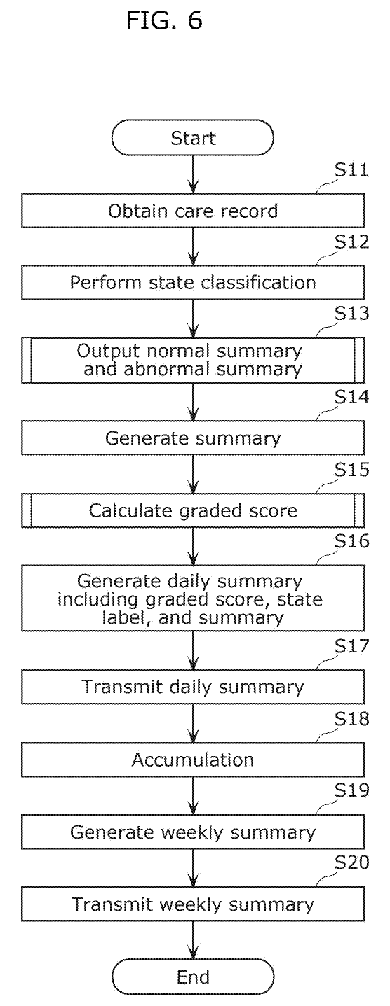

Next, the operation of information management server 10 configured as described above will be described with reference to FIG. 6 to FIG. 20. First, the operation of generating a summary related to the physical condition of subject 50 will be described with reference to FIG. 6 to FIG. 16. FIG. 6 is a flowchart indicating the first operation (assistance method) performed by information management server 10 according to the present embodiment.

As illustrated in FIG. 6, transceiver 151 of assistance processor 15 obtains a care record of subject 50 from care record collector 25 (S11). Transceiver 151 obtains, for example, a care record in the first period. Transceiver 151 periodically obtains the care record of subject 50, but the obtainment timing is not particularly limited.

Subsequently, state classifier 154 executes state classification of subject 50 based on text data included in the care record (S12). In step S12, whether subject 50 is in abnormal condition may be determined based on the text data, using a natural language processing model, and when subject 50 is in abnormal condition, the type of the abnormal condition may be also determined based on the text data, using the natural language processing model. It can be said that state classifier 154 classifies the type of a normal condition or an abnormal condition based on the text data.

State classifier 154 also obtains an attention value assigned (calculated) within the natural language processing model.

For example, state classifier 154 performs document decomposition of a text based on characters or character strings that separate meanings, to extract words from a care record. A word is a linguistic unit that consists of one or more morphemes, and is also referred to as a vocabulary. A word is, for example, the smallest unit of meaningful expression, and may be a group of phonemes extracted by dividing the characters up to the point where further division would no longer be meaningful, or may be the smallest unit that can be uttered independently. A collection of words forms phrases, clauses, and sentences. Any known method may be used for decomposing a document.

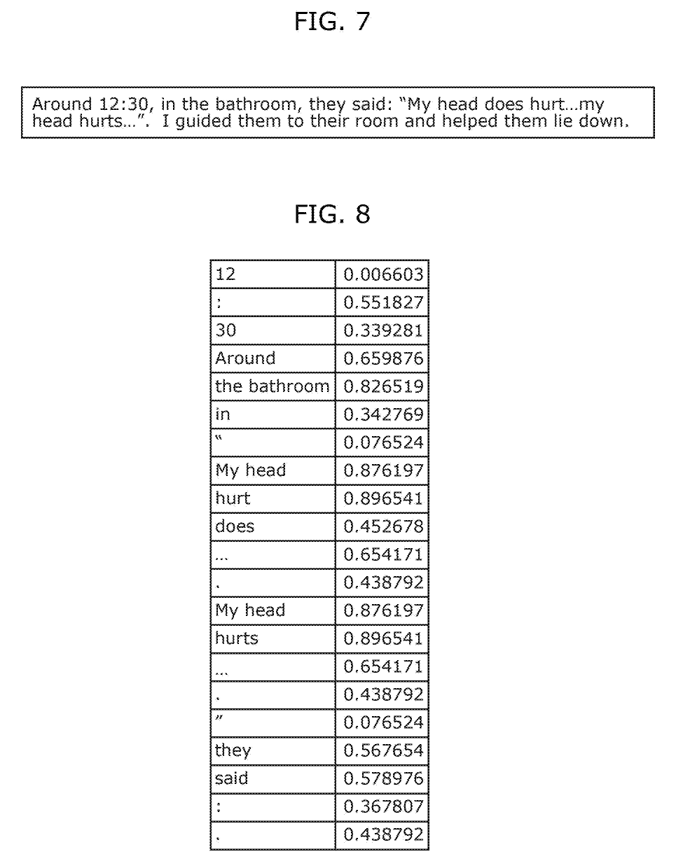

FIG. 7 is a diagram illustrating original sentences in a care record according to the present embodiment. FIG. 8 is a diagram illustrating attention values assigned on a per part basis according to the present embodiment.

FIG. 7 illustrates original sentences in a care record before document decomposition, and the left row in FIG. 8 indicates results obtained by dividing the original sentences in FIG. 7 into parts. FIG. 8 illustrates an example of dividing the original sentences in FIG. 7 into twenty-one parts.

State classifier 154 obtains an attention value on a per part basis. The attention value is a numerical value indicating the degree of contribution of each part to a classification result (e.g., a normal label, or an abnormal label indicating, for instance, restless) obtained by state classifier 154. The attention value may be a value normalized to a value greater than or equal to 0 and smaller than or equal to 1.

Numerical values in the right row in FIG. 8 indicate attention values of normalized parts. When state classifier 154 determines that subject 50 is restless, for example, an attention value shown in FIG. 8 indicates a degree to which the part influences the determination that subject 50 is restless.

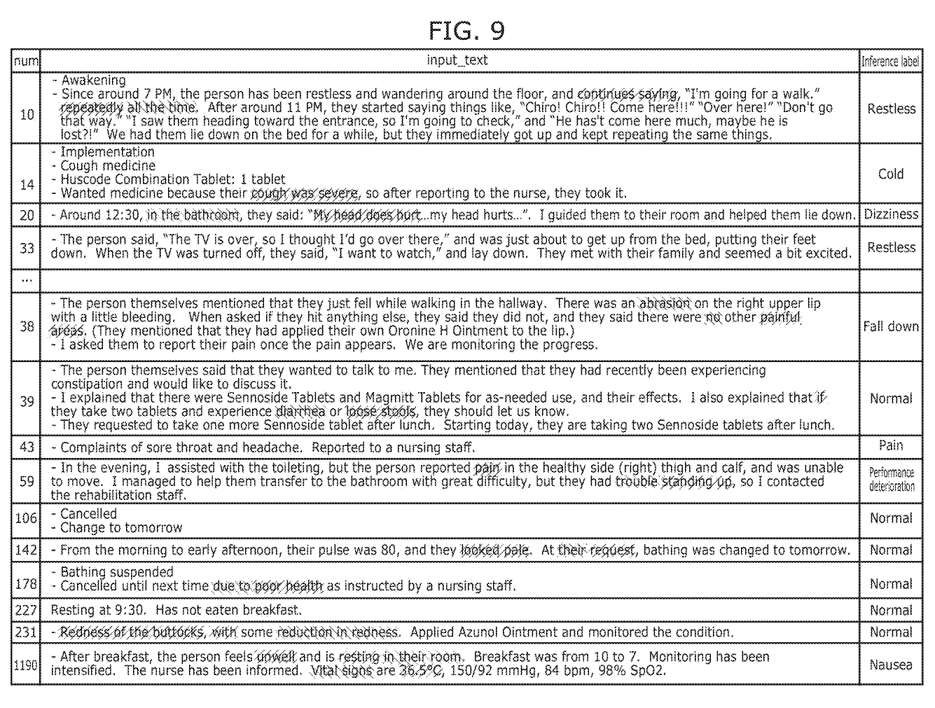

FIG. 9 is a diagram schematically illustrating the calculation result of the attention value of each text data according to the present embodiment. An inference label shown in FIG. 9 indicates the result (a normal label or an abnormal label) of classifying the text data indicated under the item “input_text”, using state classifier 154. When No. (“num” in FIG. 9) indicates 10, for example, an output obtained as a result of inputting “Awakening… repeating the same things” into the natural language processing model is “restless”. Hereinafter, inference labels are also referred to as state labels.

In FIG. 9, in the text data shown under the item “ input_text”, words with higher attention values are indicated by darker hatching and words with lower attention values are indicated by lighter hatching (or no hatching at all).

In the case where No. (“num” in FIG. 9) indicates 10, for example, “repeatedly”, “all the time”, and “continues saying” have higher contribution (e.g., attention values) in this order to the determination of the inference label as “restless”. In this case, state classifier 154 may extract, from input text data: a sentence (a divided unit sentence) including at least one of “repeatedly”, “all the time”, and “continues saying”; or a sentence (e.g., a sentence dissected by periods “.”) including at least one of these words, and output the extracted sentence to abnormal section extractor 155b.

Since a care record includes a section that is not a text record such as a numerical value and a text part unnecessary for state classification, state classifier 154 may extract only a text part necessary for state classification from text data and input the extracted text part into the natural language processing model.

For example, set phrases or descriptions that follow title words frequently appearing in care records, such as “near-miss accidents” and “condition observations”, may be excluded from text data. For example, state classifier 154 may extract from a care record a description that is likely to contain a record related to an abnormal condition, and input the extracted description into the natural language processing model.

State classifier 154 may extract from the care record predetermined items, e.g., descriptions that follow title words such as “special notes in care records”, “observation details”, “condition observations”, “response records”, “today’s condition”, “temporary medication administration”, “consultation and communication”, and “accident records”, or the surrounding descriptions, as items indicating the condition of subject 50, and input the extracted descriptions into the natural language processing model.

State classifier 154 may extract descriptions that may indicate abnormal conditions in predetermined activities of subject 50, such as eating, bathing, and toileting, and may input the extracted descriptions into the natural language processing model. Since meals, bathing, and toileting are often recorded in a care record even if there are no abnormal conditions in these activities, it is preferable to extract entries that describe any changes in condition or any discontinuation.

State classifier 154 may input text data related to consultations excluding regular check-ups into the natural language processing model. The text data may include excerpts from medical examination reports on urgent visits or unplanned visits, or excerpts that were extracted as potentially indicating abnormal conditions from medical examination reports. The process of extracting necessary excerpts from such text data is also referred to as an extraction process.

Referring again to FIG. 6, normal section extractor 155a generates and outputs a normal summary that is a summary related to a normal condition, based on normal data that is a sentence determined as describing a normal condition by state classifier 154, and abnormal section extractor 155b generates and outputs an abnormal summary that is a summary related to an abnormal condition, based on abnormal data that is a sentence determined as describing an abnormal condition by state classifier 154 (S13). When first summary generator 156 generates a daily summary, a normal summary or an abnormal summary may be generated based on care records for one day in step S13.

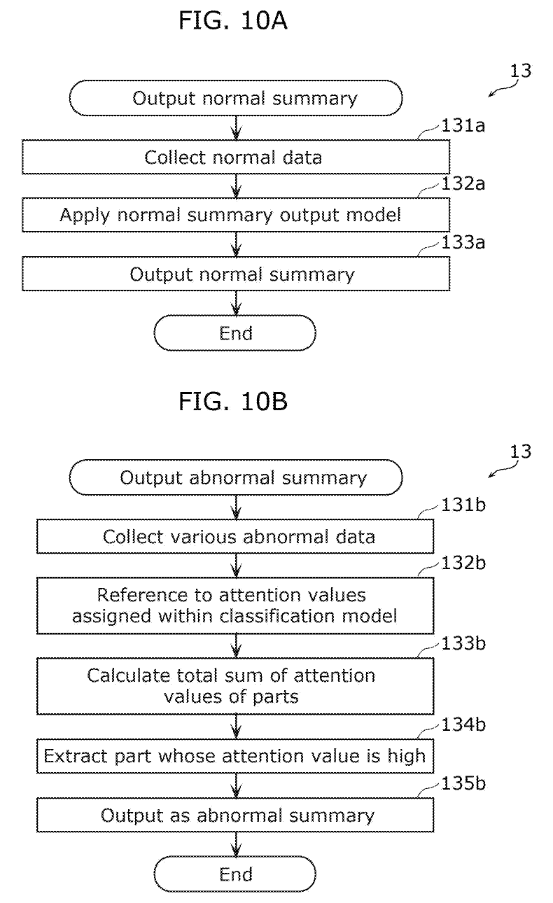

FIG. 10A and FIG. 10B are each a flowchart illustrating one example of detailed operation (an assistance method) in step S13 shown in FIG. 6. FIG. 10A illustrates an operation executed by normal section extractor 155a in step S13, and FIG. 10B illustrates an operation executed by abnormal section extractor 155b in step S13.

As illustrated in FIG. 10A, normal section extractor 155a collects normal data (S131a). For example, normal section extractor 155a may collect normal data from state classifier 154. In step S131a, sentences each determined as describing a normal condition by state classifier 154 are extracted.

Next, normal section extractor 155a applies a normal summary output model (S132a). Normal section extractor 155a inputs sentences each determined as describing an abnormal condition by state classifier 154 and summarizes the input sentences, using the normal summary output model. With this, a normal summary including at least one of the sentences each determined as describing a normal condition is generated.

Subsequently, normal section extractor 155a outputs the generated normal summary to first summary generator 156 (S133a).

As illustrated in FIG. 10B, abnormal section extractor 155b collects various types of abnormal data (S131b). Abnormal section extractor 155b collects abnormal data that is a sentence determined as describing an abnormal condition irrespective of the type of the abnormal condition. For example, abnormal section extractor 155b may collect abnormal data from state classifier 154. In step S131b, sentences each determined as describing an abnormal condition by state classifier 154 are extracted.

Next, abnormal section extractor 155b then refers to attention values assigned within a classification model (the natural language processing model in the present embodiment) (S132b) and calculates the total sum of the attention values of the parts (S133b). Abnormal section extractor 155b obtains, for example, the attention value of each part as shown in FIG. 8 and calculates the total sum of the attention values of the parts. Stated differently, abnormal section extractor 155b calculates an attention value as the part.

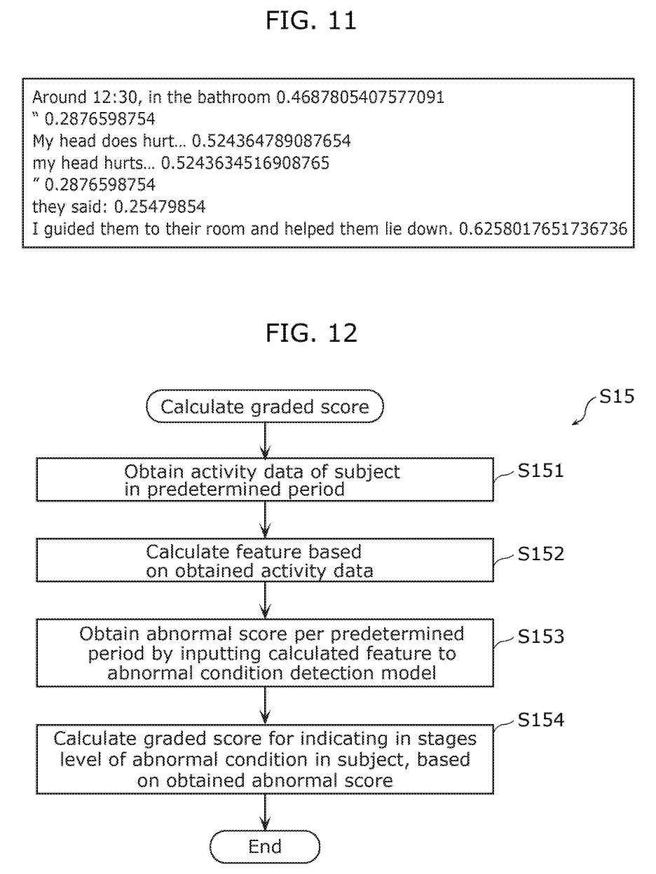

FIG. 11 is a diagram illustrating the attention value of each part according to the present embodiment.

The left row in FIG. 11 shows a result obtained by dividing the original sentences in FIG. 7 into parts. FIG. 11 shows an example in which the original sentences are divided into seven parts.

Numerical values on the right side in FIG. 11 indicate the attention values of the parts. FIG. 11 illustrates an example in which the part “I guided them to their room and had them lie down” contribute the most in the original sentences to an abnormal label obtained by inputting the original sentences into the natural language processing model. It can be said that the part is a part with a high degree of attention to the abnormal label.

Note that it can be said that the attention value used herein is a numerical value that indicates how much attention a particular part has received (how much the particular part has given an influence to the determination result) in natural language processing using natural language processing models, and indicates importance related to an abnormal condition in subject 50.

Note that the above has described that abnormal section extractor 155b calculates the attention values of one or more parts included in a sentence by summing up the attention values of the one or more parts in the sentence, but may calculate the attention value of the sentence using, for example, the mean, median, or mode of the attention values of the parts included in the sentence.

Referring again to FIG. 10B, abnormal section extractor 155b extracts a part with a high attention value among parts (S134b). Abnormal section extractor 155b may extract a part whose attention value is a predetermined value or greater among the parts, or parts with attention values in the top specified number. Note that an extracted sentence with a high attention value is one example of information based on the type of an abnormal condition in a subject.

Subsequently, abnormal section extractor 155b outputs the extracted sentence with a high attention value to first summary generator 156 as an abnormal summary (S135b).

Referring again to FIG. 6, first summary generator 156 generates a summary based on the obtained normal summary and abnormal summary (S14). First summary generator 156 generates, for example, a summary including: a normal summary; and a sentence resulting from complementing parts in the abnormal summary with a high attention value with particles and other elements to connect the parts naturally in Japanese. The summary generated in step S14 is one example of the first summary.

First summary generator 156 may cause storage 157 to store the generated summary. First summary generator 156 may also transmit the generated summary to display terminal 30 over communication network 40. With this, a summary summarizing how subject 50 spent that day can be presented to user 61. User 61 can get a general idea of the condition of subject 50 just by reviewing the summary, without having to check a care record that contains a large amount of text data. This can prevent user 61 from overlooking important items.

In this way, assistance processor 15 divides text data into parts, calculates the attention value of each of the parts, and generates a first summary based on the attention value of each part.

Assistance processor 15 then calculates a graded score for subject 50 based on the activity data of subject 50 obtained by sensor 20 (S15).

FIG. 12 is a flowchart illustrating detailed operation (assistance method) in step S15 shown in FIG. 6.

As illustrated in FIG. 12, transceiver 11 obtains the activity data of subject 50 in a predetermined period (e.g., activity data including at least one of respiratory rate or heart rate) (S151). The predetermined period is, for example, a first period.

Subsequently, feature calculator 13 calculates a feature based on the activity data obtained in step S151 (S152). Feature calculator 13 calculates, for example, a feature per hour. Feature calculator 13 may calculate, for example, hourly features for the target day on which the physical condition of subject 50 is to be detected, based on activity data that includes at least the respiratory rate or heart rate of subject 50 obtained by transceiver 11. Note that the number of features to be calculated is not particularly limited and may be one or plural.

Subsequently, score calculator 14 obtains an abnormal score per predetermined period by inputting the feature calculated in step S152 into an abnormal condition detection model trained in advance (S153). Score calculator 14 calculates an abnormal score per hour from features per hour, for example. Score calculator 14 obtains an abnormal score indicating the degree of an abnormal condition per hour in a predetermined period including the target day by inputting the feature calculated by feature calculator 13 into the abnormal condition detection model generated in advance. When the predetermined period is one day, the abnormal score of that one day is calculated based on twenty-four abnormal scores. The abnormal score of that one day is, for example, the mean, median, or mode of twenty-four abnormal scores, but is not limited to this example.

Subsequently, score calculator 14 calculates a graded score for indicating, in stages, the degree of the abnormal condition in subject 50, based on the abnormal score obtained in step S153 (S154). Score calculator 14 calculates, for example, the mean value of abnormal scores per day from abnormal scores per hour. In the present embodiment, score calculator 14 calculates the mean value of abnormal scores per day in a predetermined period including the day on which the physical condition of subject 50 is to be detected, based on abnormal scores per hour.

Score calculator 14 calculates a stepped threshold by calculating the mean and standard deviation of past abnormal scores for the target date (e.g., abnormal scores for approximately the past 90 days).

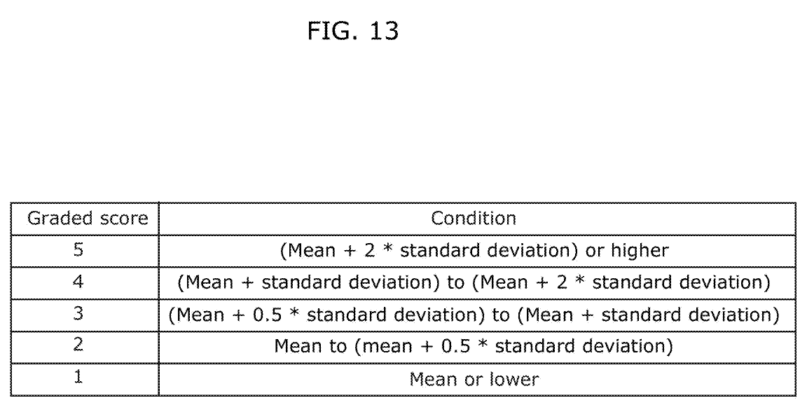

FIG. 13 is a diagram illustrating one example of five-level graded scores and conditions for the graded scores according to the present embodiment.

As illustrated in FIG. 13, when calculating a five-level graded score, for example, score calculator 14 can calculate a threshold using a mean and standard deviation. For example, a threshold for the graded score indicating 1 is at least a mean based on the condition indicated in FIG. 13, and a threshold for the graded score indicating 2 includes a value obtained by adding half a standard deviation to the mean and the mean.

Score calculator 14 then calculates a graded score by applying the calculated stepped threshold to the mean value of abnormal scores of the target day. More specifically, score calculator 14 calculates the value of the graded score by determining the mean value of the abnormal scores of the target day using the threshold calculated based on the condition as shown in FIG. 13.

Score calculator 14 may also verify whether a value whose graded score calculated in step S154 indicates 4 or 5, i.e., whether a value indicating the presence of an abnormal condition is calculated. When the graded score is 4 or 5, factor analysis may be performed on each of elements such as heart rate, respiratory rate, out-of-bed-rate, body temperature, and food intake included in activity data used for calculating features.

By calculating the scores as described above, it is possible to detect small changes in the physical condition of subject 50 that may lead to an abnormal condition in subject 50 (i.e., early signs of an abnormal condition).

Referring again to FIG. 6, first summary generator 156 generates a daily summary including a graded score, a state label (an inference label), and a summary (S16), and transmits the daily summary to display terminal 30 over communication network 40 (S17). The summary generated in step S14 may be transmitted as a daily summary. Step S17 is one example of outputting presentation information that is based on the result of determining whether subject 50 has an abnormal condition. Information indicating a daily summary is one example of presentation information.

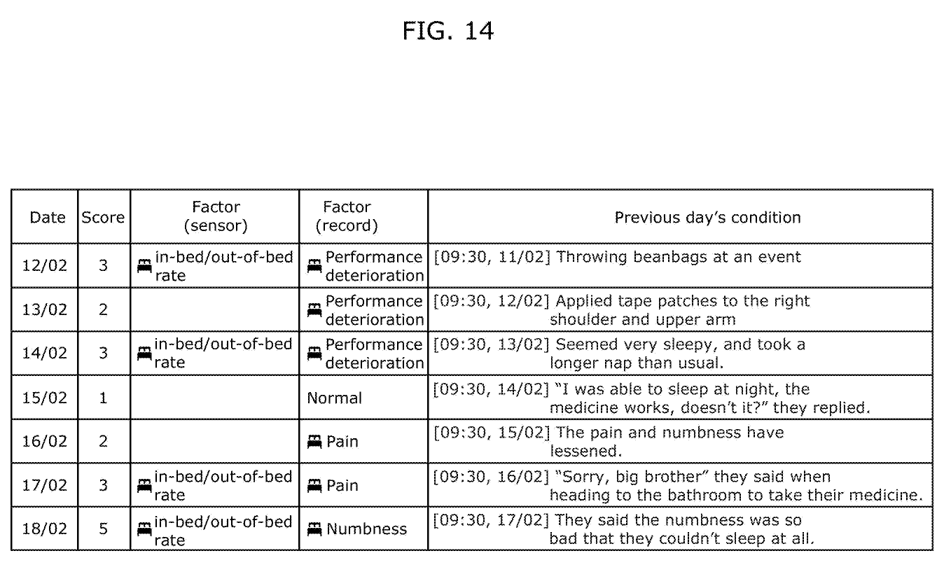

FIG. 14 is a diagram illustrating one example of a daily summary including a score according to the present embodiment. FIG. 14 illustrates one example of a daily summary generated in step S16. A daily summary needs to include at least the date of the daily summary and a previous day’s condition. The previous day’s condition corresponds to a summary generated in step S14. For example, presentation information needs to include at least a summary generated in step S14. Alternatively, presentation information may include at least a summary generated in step S14 or state classification results (factors (records) shown in FIG. 14). FIG. 14 illustrates the daily summaries of subject 50 for one week from February 12 to February 18.

As illustrated in FIG. 14, a daily summary includes a date, a score (here, a graded score), a factor (sensor), a factor (record) and a previous day’s condition. For example, the table shown in FIG. 14 is presented by display terminal 30.

A date is a target date for which a score or the like is calculated.

A score is a graded score calculated in step S154, but may be, for example, an abnormal score obtained in step S153. A graded score and an abnormal score are examples of scores based on abnormal scores.

A factor (sensor) indicates the activity data of subject 50 obtained by sensor 20. In the example in FIG. 14, the in-bed/out-of-bed rate is shown. The value of the factor (sensor) may be recorded only for a day with a score that is a predetermined value or greater (3 or greater in the example in FIG. 14). The factor (sensor) may include, for example, information indicating a factor with a high score conceivable from sensor data when the score is the predetermined value or greater.

A factor (record) indicates a state label. In other words, the factor (record) indicates a classification result obtained by state classifier 154.

A previous day’s condition is a description that might lead to changes observed today and is extracted from the previous day’s care record. Today’s condition may be described regardless of the previous day. The previous day’s condition is information that forms the basis for which a score is calculated.

In this way, by presenting the score together with the basis for calculating the score, it is possible to concretize the “meaning of the score”. Presenting such a daily summary makes it possible for user 61 to correctly understand the score and use it as a hint for subsequent actions. For example, presenting, with the score, a factor (record) indicating the state of a care record and a summary generated from the text data of the care record (a previous day's condition in the example in FIG. 14) in chronological order can encourage behavioral changes in user 61 more effectively than when only the score is displayed, thereby enabling proactive care. Additionally, since scores and other information are presented in chronological order, user 61 can understand the condition of subject 50 including how it looked a little while ago, which can help prevent overlooking symptoms related to the physical condition of subject 50.

Subsequently, first summary generator 156 stores the generated daily summary in storage 157 (S18). With this, daily summaries are accumulated in storage 157.

Note that steps S11 to S18 are repeatedly executed for each first period (e.g., each day).

Subsequently, second summary generator 158 generates a summary of care records in a second period longer than the first period, based on daily summaries stored in storage 157. The summary of the care records in the second period is generated based on summaries of care records in the first periods. In the present embodiment, second summary generator 158 generates a weekly summary based on daily summaries for one week (S19).

Second summary generator 158, for example, extracts more notable descriptions from a week’s worth of daily summaries, and generates a weekly summary that includes the extracted descriptions. Second summary generator 158 generates a weekly summary based on, for example, the attention value of each sentence in the week’s worth of daily summaries. Second summary generator 158, for example, extracts a sentence whose attention value is a predetermined value or greater, or sentences with attention values in the top specified number from the week’s worth of daily summaries, and generates a weekly summary based on the extracted one or more sentences. Information indicating the weekly summary is one example of presentation information.

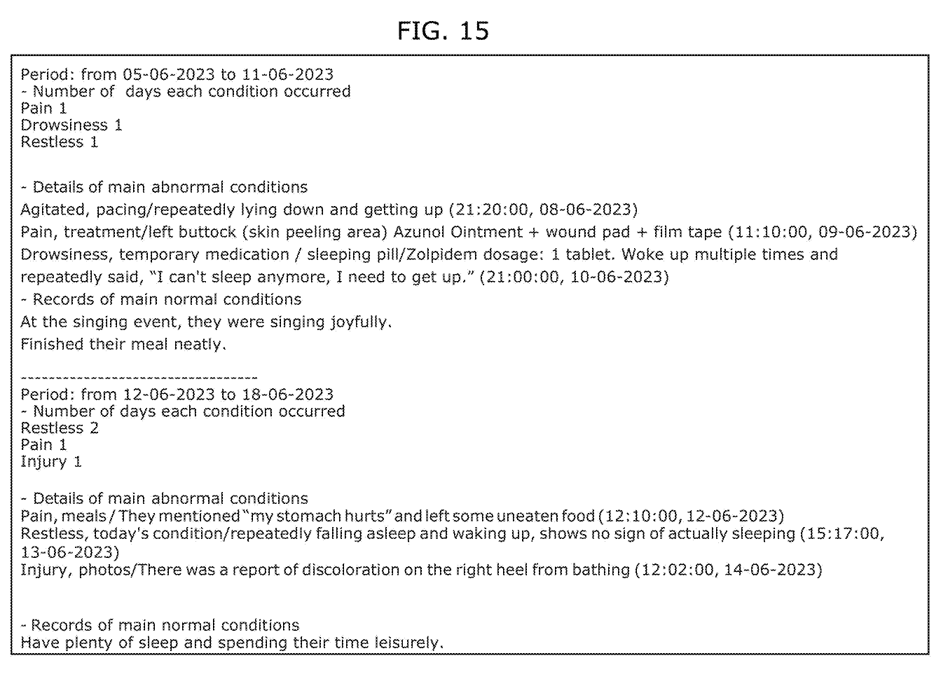

FIG. 15 is a diagram illustrating one example of a weekly summary according to the present embodiment.

As illustrated in FIG. 15, a weekly summary includes the number of days each condition (abnormal label) occurred, the details of main abnormal conditions, and the records of main normal conditions.

The number of days each condition occurred indicates the number of times (days) the condition happened in a week for each abnormal condition.

The details of main abnormal conditions are the details of abnormal conditions extracted from a daily summary based on attention values.

The records of main normal conditions are the details of normal conditions extracted from a daily summary.

By presenting such a weekly summary to user 61, user 61 can get an overview of the condition of subject 50 over the past week and can also utilize the weekly summary in a care plan for the following week. Additionally, since necessary information is extracted and presented from a large amount of care records for a week, user 61 can easily know the health condition of subject 50 for the week. Furthermore, because the weekly summary summarizes the condition of subject 50 over the week, the time required for user 61 to create reports on the user's status, reports for the family of subject 50, or the like can be reduced.

In addition, when second summary generator 158 obtains activity data obtained by sensor 20 sensing subject 50 for that week, the activity data for that week may be also included in the weekly summary. For example, second summary generator 158 may include, in the weekly summary, a week's worth of activity data or activity data of a day on which an abnormal condition occurred in subject 50.

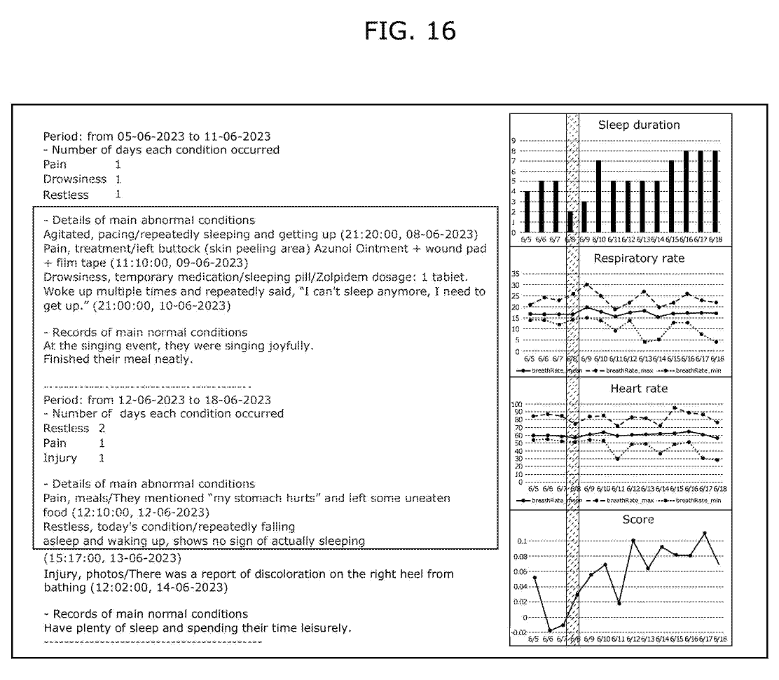

FIG. 16 is a diagram illustrating one example of a weekly summary including activity data according to the present embodiment. Information indicating the weekly summary, which is shown in FIG. 16, including activity data is one example of presentation information.

As illustrated in FIG. 16, the weekly summary may include activity data for one week in addition to a summary. The activity data is displayed in a graph showing changes over time, but may also be displayed using numerical values. Activity data includes, for example, sleep duration, respiratory rate, and heart rate, but may also include other activity data.

The weekly summary may include a graph showing changes over time in scores for a week (e.g., graded scores calculated in step S15).

In the graph, data obtained on a day when an abnormal condition occurred may be highlighted. The day when an abnormal condition occurred refers to, for example, a day when an abnormal condition mentioned in the weekly summary occurred. When abnormal conditions occurred during a week, for example, data obtained on the day when a particularly important abnormal condition occurred may be highlighted.

Subsequently, second summary generator 158 transmits the weekly summary generated in step S19 to display terminal 30 over communication network 40 (S20). This allows the weekly summary to be presented to user 61. Step S20 is an example of outputting presentation information that is based on the result of determining whether subject 50 has an abnormal condition.

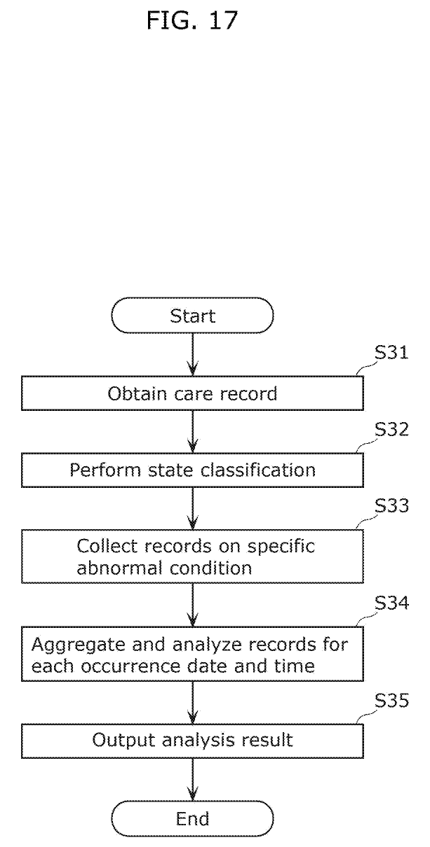

Next, the operation of aggregating abnormal labels for subject 50 will be described with reference to FIG. 17 to FIG. 20. FIG. 17 is a flowchart illustrating a second operation (an assistance method) performed by information management server 10 according to the present embodiment. Steps S31 and S32 shown in FIG. 17 are the same as steps S11 and S12 shown in FIG. 6, and description is omitted.

As illustrated in FIG. 17, aggregator 159 collects a record of a specific abnormal condition (a target label) among abnormal conditions associated with subject 50 (S33). The specific abnormal condition may be set by, for instance, user 61. The record of each of the abnormal conditions associated with subject 50 is stored in storage 157, and aggregator 159 may read out the record of a specific abnormal condition from storage 157 in step S33.

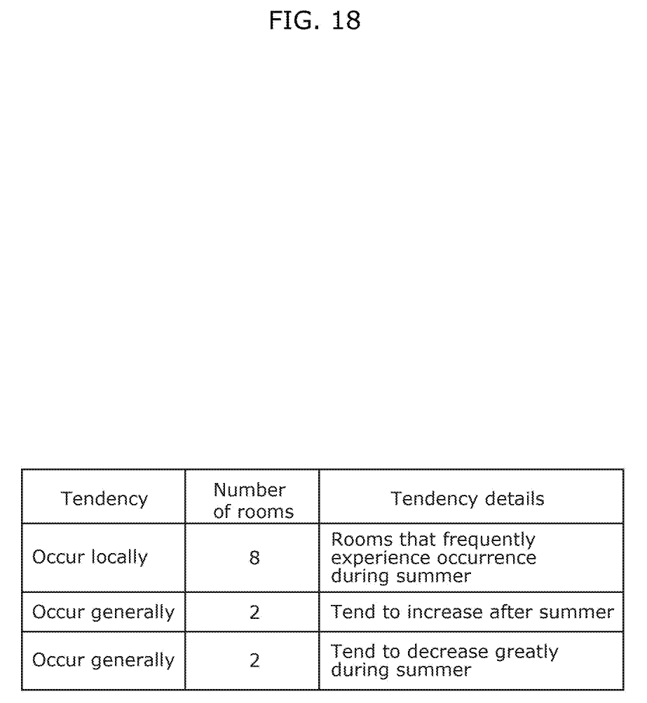

Subsequently, aggregator 159 aggregates and analyzes the number of occurrences for each date and time of occurrence of an abnormal condition included in the collected abnormal records (S34), and outputs the analysis result (aggregation result) to display terminal 30 (S35). Aggregating the number of occurrences for each date and time when an abnormal condition occurred is one example of aggregating the date and time when an abnormal condition occurred. Information based on the aggregation result is information shown in FIG. 18 to FIG. 20 described below, and is one example of presentation information.

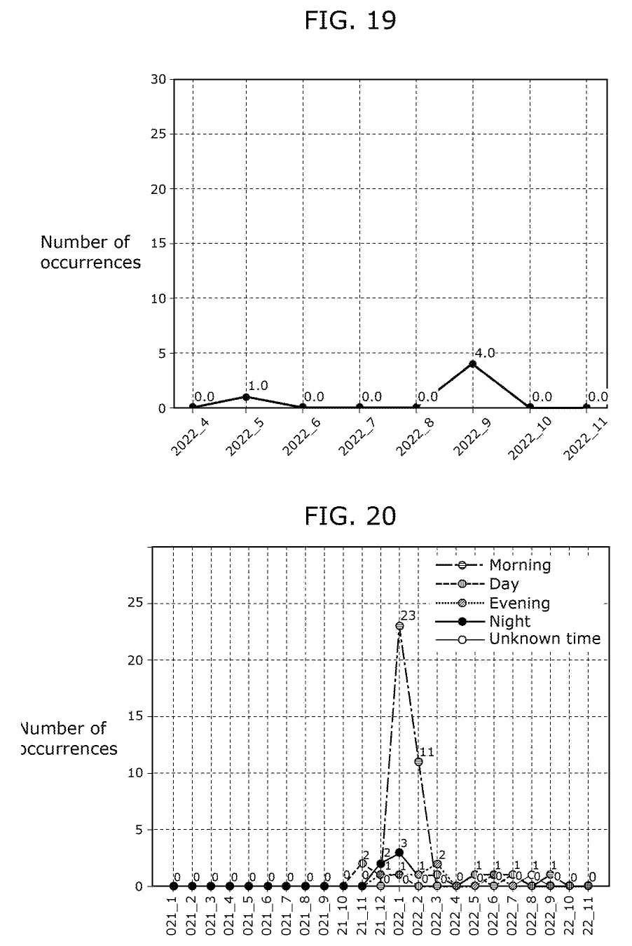

FIG. 18 to FIG. 20 are diagrams illustrating examples of aggregation information according to the present embodiment. FIG. 18 shows, for example, the results of analyzing the overall tendency of abnormal conditions in an introduction facility (e.g., a nursing care facility). There may be one subject 50 or a plurality of subjects 50 in each room. FIG. 19 and FIG. 20 show the results of analyzing aggregated data of subjects 50.

As illustrated in FIG. 18, an analysis result includes the tendency of an abnormal condition, the number of rooms, and tendency details.

The tendency shows whether a certain abnormal condition occurs locally or generally within the introduction facility. In other words, the tendency shows a state in which the abnormal condition occurred at that point in time. The expression “occurs locally” means that the abnormal condition tends to occur in only a few rooms (i.e., among a small number of people) within the introduction facility, indicating a bias in the occurrence of the abnormal condition.

The number of rooms indicates the number of rooms where the abnormal condition occurred.

The tendency details indicate the tendency of conditions in which the abnormal condition occurred when viewed in chronological order.

For example, on the top tier of the three tiers, there are eight rooms in the introduction facility where an abnormal condition (an abnormal label) that tends to occur locally was observed, which shows that the abnormal condition tends to be more frequent in rooms during the summer. On the second tier of the three tiers, there are two rooms where an abnormal condition (an abnormal label) that tends to occur generally was observed, which shows that the abnormal condition tends to increase after the summer.

FIG. 19 shows the result of aggregating the number of occurrences of an abnormal condition by month. In the example in FIG. 19, a specific abnormal condition occurs frequently in September. This indicates that, for subject 50, there is a tendency for this specific abnormal condition to occur more easily in September. By displaying (visualizing) the graph shown in FIG. 19 on display terminal 30 of user 61, it is possible to inform user 61 of an analysis result regarding which abnormal condition is more likely to occur in which season for subject 50. The graph shown in FIG. 19 is one example of information indicating the influence of seasons on the occurrence of a specific abnormal condition.

FIG. 20 shows the result of aggregating the number of occurrences of an abnormal condition occurring each time period and each month. Time periods include morning, afternoon, evening, night, and unknown time. This indicates that for subject 50, there is a tendency for a specific abnormal condition to occur frequently on winter mornings. By presenting (visualizing) the graph shown in FIG. 20 on display terminal 30 of user 61, it is possible to inform user 61 of an analysis result regarding which abnormal condition is likely to occur in which time period for subject 50. The graph shown in FIG. 20 is one example of information indicating the influence of time on the occurrence of a specific abnormal condition.

In this way, since the tendency of occurrence of an abnormal condition that differs for each subject 50 can be known, user 61 will be able to respond to subject 50 efficiently.

Note that the aggregation shown in FIG. 19 and FIG. 20 is performed for each subject 50.

In addition, it is sufficient if at least one of the table shown in FIG. 18, the graph shown in FIG. 19, or the graph shown in FIG. 20 is presented to user 61 as presentation information.

### Variation of the Embodiment

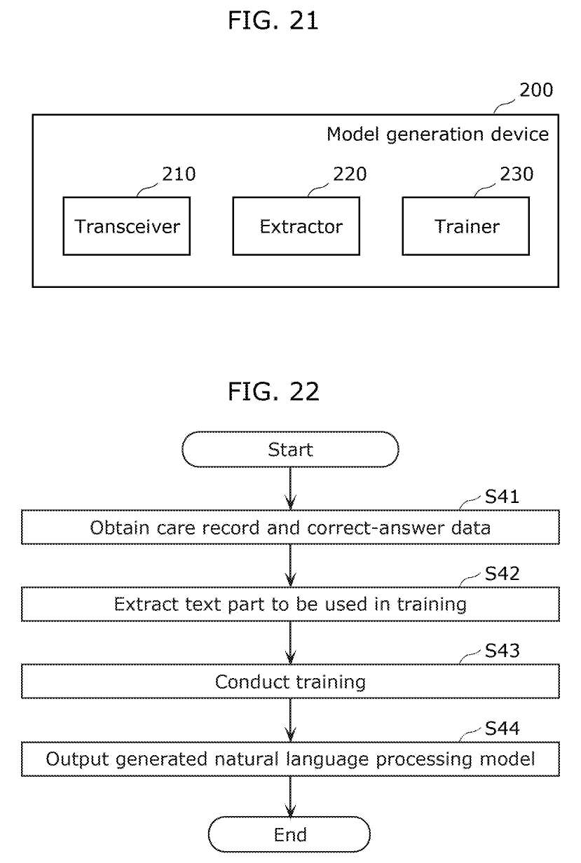

In the present variation, the generation of the machine learning model used in the embodiment will be described with reference to FIG. 21 and FIG. 22.

FIG. 21 is a block diagram illustrating one example of the functional configuration of model generation device 200 according to the present variation.

As illustrated in FIG. 21, model generation device 200 includes transceiver 210, extractor 220, and trainer 230. Model generation device 200 is implemented by, for example, a computer including, for instance, a processor (microprocessor), memory, and a communication interface.

Transceiver 210 includes, for example, a communication interface, and transmits and receives various types of information between care record collector 25 and information management server 10 over communication network 40. For example, transceiver 210 obtains a care record of subject 50 in a predetermined period from care record collector 25. For example, transceiver 210 obtains, from display terminal 30 or other terminal device, correct-answer data (label data) indicating a normal condition or an abnormal condition (e.g., the type of an abnormal condition) for text data included in the obtained care record. For example, transceiver 210 outputs the machine learning model generated by model generation device 200 to information management server 10.

Extractor 220 extracts a text part for use in training from the text data included in the care record. Extractor 220 extracts only a text part necessary for training from the text data. Extractor 220 may, for example, exclude, from the text data, standard expressions or descriptions that follow title words frequently appearing in a care record, such as “near-miss accidents” and “condition observations”. For example, extractor 220 may extract from the care record descriptions that are likely to contain records related to abnormal conditions.

Extractor 220 may extract, from the care record, descriptions that follow title words such as “special notes”, observation content”, “condition observations”, “response records”, “today’s condition”, “temporary medication administration”, “consultation and communication”, and “accident records”, or the surrounding descriptions in the care record, as items indicating the condition of subject 50.

Extractor 220 may also extract, for example, a description that implies the occurrence of an abnormal condition in predetermined activities of subject 50, such as eating, bathing, and toileting. Even if there are no abnormal conditions in meals, bathing, or toileting, these are often recorded in a care record, so it is preferable to extract a description that indicates any changes in condition or any discontinuation.

Trainer 230 trains a machine learning model using the extracted text part and the correct-answer data. The training method is not particularly limited and any known method may be used. For example, trainer 230 may update the parameters of the machine learning model through back propagation based on prediction errors between the text part and the correct-answer data serving as a true value. In other words, trainer 230 performs training of updating the parameters so that errors (difference) between the true value and the correct-answer data are the smallest. In this way, trainer 230 performs training by performing back propagation training between the text part and the correct-answer data.

Next, a model generation method employed by model generation device 200 configured as above will be described with reference to FIG. 22. FIG. 22 is a flowchart illustrating the operation (assistance method) of generating a machine learning model according to the present variation. FIG. 22 illustrates the generation of a natural language processing model.

As illustrated in FIG. 22, transceiver 210 obtains correct-answer data indicating a care record including text data and the result of determining whether subject 50 has an abnormal condition (S41).

Subsequently, extractor 220 extracts a text part for use in training from the text data included in the obtained care record (S42). For example, extractor 220 may extract, as the text part, descriptions that are likely to contain records related to abnormal conditions. Step S42 is preprocessing before training a machine learning model.

Subsequently, trainer 230 trains a natural language processing model, which is one example of the machine learning model, using the extracted text part and the correct-answer data (S43).

Subsequently, transceiver 210 outputs the natural language processing model generated by trainer 230 to information management server 10 (S44).

Note that the process in step S42 may be omitted.

### Other Embodiments

An assistance method and others according to one aspect or plural aspects of the present disclosure have been described based on the embodiment, etc., but the present disclosure is not limited to this embodiment, etc. Forms obtained by making various modifications to the present embodiment that persons skilled in the art may conceive or forms obtained by combining elements from different embodiments may be included in the present disclosure so long as they do not depart from the essence of the present disclosure.

The machine learning model described in the aforementioned embodiment is a neural network (NN), and may be, for example, a convolution neural network (CNN), a recurrent neural network (RNN), or a long-short term memory (LSTM).

The aforementioned embodiment has described an example in which the normal summary output model and the abnormal section summary model are machine learning models, but these models are not limited to this example and may be implemented by training models other than such machine learning models. The normal summary output model and the abnormal section summary model are examples of learning models.

The aforementioned embodiment has illustrated an example in which some of the machine learning models are supervised models but the machine learning models may be unsupervised models.

The aforementioned embodiment has illustrated an example in which the information management server and the model generation device are separate bodies, but they may be implemented as a single device. For example, the information management server may include at least one of functions of the model generation device (e.g., all of the functions).

In the aforementioned embodiment, each element may be configured by dedicated hardware or implemented by executing a software program suitable for the element. Each element may be implemented by a program executor, such as a CPU or a processor, reading and executing a software program recorded on a recording medium such as a hard disk or semiconductor memory.

An order in which each of steps in a flowchart is executed is for explaining the present disclosure in detail and may be an order other than this order. Some of the steps may be executed simultaneously (in parallel) with other steps, or some of the steps may not be executed.

Division of function blocks in a block diagram is one example, and a plurality of function blocks may be implemented as one function block, or one function block may be divided into a plurality of function blocks, or some functions may be moved to other function blocks. The functions of a plurality of function blocks with similar functions may be processed in parallel or time-division by single hardware or software.

The information management server according to the aforementioned embodiment may be implemented as a single device or using a plurality of devices. When the information management server is implemented using a plurality of devices, each component of the information management server may be distributed across the plurality of devices in any manner. When the information management server is implemented using a plurality of devices, the method of communication between these devices is not particularly limited and may be wireless communication or wired communication. In addition, between devices, a combination of wireless communication and wired communication may also be used.

Each of the elements described in the aforementioned embodiment may be implemented as software or as large-scale integration (LSI) that is typically an integrated circuit. These may take the form of individual chips, or may be partially or entirely packaged into a single chip. Although the term “LSI” is used here, other names, such as IC, system LSI, super LSI, ultra LSI may be used, depending on the level of integration. The method for implementing the integrated circuit is not limited to LSI; the circuit may be implemented using a dedicated circuit (a generic circuit that executes a dedicated program), a generic processor, or the like. A field programmable gate array (FPGA) capable of post-production programming or a reconfigurable processor in which the connections and settings of the circuit cells within the LSI can be reconfigured may be used as well. Furthermore, should technology for implementing integrated circuits that can replace LSI appear due to advancements in semiconductor technology or the appearance of different technologies, the integration of the above functions may be performed using such technology.

“System LSI” refers to very-large-scale integration manufactured by integrating a plurality of processing units on a single chip, and specifically, refers to a computer system configured including a microprocessor, read only memory (ROM), random access memory (RAM), and the like. A computer program is recorded in the ROM. The system LSI circuit realizes the functions by the microprocessor operating in accordance with the computer program.

One aspect of the present disclosure may be a computer program that causes a computer to execute each characteristic step included in the assistance method described with reference to any one of FIG. 6, FIG. 7, FIG. 12, FIG. 17, and FIG. 22.

The program may be, for example, a program for causing a computer to execute the assistance method. One aspect of the present disclosure may be a computer-readable non-transitory recording medium on which such a program is recorded. For example, such a program may be recorded on a recording medium and distributed or circulated. For example, by installing a distributed program on a device with another processor and having that processor execute the program, it becomes possible to have the device perform the respective processes mentioned above.

### Industrial Applicability

The present disclosure is useful for, for instance, assistance methods for assisting users to know the health conditions of subjects that are based on care records.

1. An assistance method for assisting a user to know a health condition of a subject, the assistance method comprising:
obtaining a care record of the subject in a first period, the care record including text data;
determining whether the subject has an abnormal condition based on the text data, using a natural language processing model; and
outputting presentation information to an information terminal of the user, the presentation information being based on a result of the determining.

2. The assistance method according to claim 1, further comprising:
determining, when the subject has an abnormal condition, a type of the abnormal condition based on the text data, using the natural language processing model, wherein
the presentation information includes information based on the type of the abnormal condition determined.

3. The assistance method according to claim 2, further comprising:
extracting at least one of the following from the care record, using a learning model: (i) a normal section describing that the subject is in normal condition; or (ii) an abnormal section describing that the subject is in abnormal condition; and
generating a first summary describing the health condition of the subject in the first period, based on the at least one of the normal section or the abnormal section extracted, wherein
the presentation information includes the first summary.

4. The assistance method according to claim 3, wherein
in the generating of the first summary: the text data is divided into parts; an attention value of each of the parts is calculated, the attention value indicating a degree to which the part contributes to a result of the determining of the type of the abnormal condition; and the first summary is generated based on the attention values of the parts.

5. The assistance method according to claim 4, wherein
in the generating of the first summary, at least one part whose attention value is a first predetermined value is extracted from among the parts, and the first summary is generated based on the at least one part extracted.

6. The assistance method according to claim 5, further comprising:
obtaining activity data of the subject in the first period;
calculating a feature based on the activity data obtained; and
obtaining, based on the feature, an abnormal score indicating a degree of the abnormal condition per first period, wherein
the presentation information includes information that associates the first summary with a score that is based on the abnormal score.

7. The assistance method according to claim 2, wherein
a second period includes two or more first periods each of which is the first period,
the assistance method further comprises:
using a learning model, extracting at least one of the following from the care record: (i) a normal section describing that the subject is in normal condition; or (ii) an abnormal section describing that the subject is in abnormal condition;
generating a first summary describing the health condition of the subject in the first period, based on the at least one of the normal section or the abnormal section extracted; and
generating a second summary based on (i) two or more first summaries in each of the two or more first periods included in the second period, each of the two or more first summaries being the first summary, and (ii) an attention value of each of the two or more first summaries, the attention value indicating a degree to which the first summary contributes to the result of the determining of whether the subject has an abnormal condition, and
the presentation information includes the second summary.

8. The assistance method according to claim 7, wherein
in the generating of the second summary, at least one sentence whose attention value is a second predetermined value is extracted from the two or more first summaries, and the second summary is generated based on the at least one sentence extracted.

9. The assistance method according to claim 7, further comprising:
obtaining activity data of the subject obtained by sensing the subject in the second period; and
outputting time-series data of the activity data to the information terminal of the user, the time-series data being associated with the second summary.

10. The assistance method according to claim 1, further comprising:
collecting a record of a specific abnormal condition among abnormal conditions associated with the subject; and
aggregating occurrence dates and times of two or more specific abnormal conditions that have been collected, wherein
the presentation information includes information based on a result of the aggregating.

11. The assistance method according to claim 10, wherein
the information based on the result of the aggregating includes information indicating an influence of a season or time on occurrence of the specific abnormal condition.

12. The assistance method according to claim 1, further comprising:
extracting a description related to at least one of the following from the care record: a predetermined item; a predetermined activity of the subject; or a report on a consultation excluding a regular check-up, wherein
the text data includes the description extracted.

13. An assistance device for assisting a user to know a health condition of a subject, the assistance device comprising:
an obtainer that obtains a care record of the subject in a first period, the care record including text data;
a determiner that determines whether the subject has an abnormal condition based on the text data, using a natural language processing model; and
an outputter that outputs presentation information to an information terminal of the user, the presentation information being based on a result of the determination.

14. A non-transitory computer-readable recording medium for use in a computer, the recording medium having recorded thereon a computer program for causing the computer to execute the assistance method according to claim 1.
